(19) United States
(12) Patent Application Publication
(10) Pub. No.: US 2021/0371303 A1
(43) Pub. Date: Dec. 2, 2021

(54) WATER FILTER

(71) Applicants: Hsiu-Ling Yang, Taoyuan City (TW); YI CHANG HSIANG INDUSTRIAL CO., LTD., Taoyuan City (TW)

(72) Inventors: HSIU-LING YANG, Taoyuan City (TW); GARY WANG, Taoyuan City (TW)

(21) Appl. No.: 16/889,827

(22) Filed: Jun. 2, 2020

(51) Int. Cl. C02F 1/32; A61L 2/10; A61L 2/24; C02F 1/72; C02F 1/28

(52) U.S. Cl. CPC C02F 1/32; A61L 2/10; A61L 2/24; C02F 1/725; C02F 1/281; A61L 2202/11; C02F 2307/06; A61L 2202/14; C02F 2305/10; C02F 2303/04; C02F 2201/326; C02F 2201/009; A61L 2202/122

(57) ABSTRACT

A water filter includes a housing, a spoiler unit, a power generation unit, a sterilization unit and a purification unit. The housing includes an input portion, an output portion and a first thread portion. The spoiler unit is provided in the housing and corresponds to the input portion. The power generation unit is provided in the housing and is coupled to the spoiler unit. The sterilization unit is provided in the housing and is electrically connected to the power generation unit, and includes a sterilization light source. The purification unit is provided in the housing and corresponds to the output portion and the sterilization unit, and includes a plurality of purification particles. Thus, the water filter is enabled to be coupled to a faucet using the first thread portion, allowing tap water to flow toward the direction from the input portion to the output portion.

### BACKGROUND OF THE INVENTION

### 1. Field of the Invention

The present invention relates to a water filter, and more particularly to a water filter that sterilizes and purifies tap water, and eliminates impurities in the tap water.

### 2. Description of the Related Art

Tap water commonly serves the purposes of washing, cleaning and drinking in the daily lives of people, so water quality is expected to be clean to a certain extent. In addition, since drinking water directly enters the human body, an even higher water quality is demanded. To achieve an effect of water purification, people ordinarily choose to filter tap water by a filter additionally installed.

Most conventional water filters filter out impurities from the water and thus achieve deodorization by activated carbon, which however does not satisfy effects of sterilization, impurity removal and deodorization all at the same time. As a result, such common water filters are unable to meet requirements of users.

### BRIEF SUMMARY OF THE INVENTION

In view of the issues and drawbacks of the prior art, the inventor has dedicated to research and development of a water filter that is capable of sterilizing and purifying tap water and removing impurities from the tap water, hence achieving the object of dual-effect water purification.

To achieve the above and other objects, the present invention provides a water filter including a housing, a spoiler unit, a power generation unit, a sterilization unit and a purification unit. The housing includes on two ends thereof an input portion and an output portion that are in communication with each other. The input portion includes a first thread portion, enabling the input portion to be detachably coupled to a faucet using the first thread portion. The spoiler unit is provided in the housing and corresponds to the input portion. The power generation portion is provided in the housing and is coupled with the spoiler unit. The sterilization unit is provided in the housing and is electrically connected to the power generation unit, and includes a sterilization light source. The purification unit is provided in the housing and corresponds to the output portion and the sterilization unit, and includes a plurality of water purification particles.

In the foregoing water filter, the housing includes an upper housing and a lower housing. The upper housing includes a second thread portion, and the lower housing includes a third thread portion, such that the upper housing is enabled to be detachably coupled to the third thread portion of the lower housing by the second thread portion, the input portion is provided at the upper housing, and the output portion is provided at the lower housing.

In the foregoing water filter, the housing has a light transmissive area, which may correspond to the sterilization light source.

In the foregoing water filter, the housing further includes a neck portion, which is in communication with the input portion and the spoiler unit.

In the foregoing water filter, the output portion consists of a plurality of through holes that are smaller than the plurality of purification particles.

In the foregoing water filter, the power generation unit includes an electromagnetic power generator and a vane group. The van group is connected to the electromagnetic power generator. The electromagnetic power generator has a limiting groove on an upper end thereof, and a channel portion on each of both sides thereof.

In the foregoing water filter, the spoiler unit includes an assembly portion, a hood and a plurality of spoiler entrances. The assembly portion encircles the bottom of the hood. The plurality of spoiler entrances are provided on a periphery of the hood. The assembly portion is provided at the limiting slot of the electromagnetic power generator. The hood covers the vane group. The plurality of spoiler entrances are in communication with the plurality of channel portions, and are provided along the tangent direction of the vane group.

In the foregoing water filter, the sterilization unit includes a control module, at least one sterilization lamp and a waterproof glue layer. The control module is provided at the bottom of the power generation unit. The sterilization lamps are provided in an electrically connected manner on one surface of the control module, and emit a sterilization light source. The waterproof glue layer is provided on one surface of the control module so as to expose the sterilization lamps.

In the foregoing water filter, the plurality of sterilization lamps are UVC light sources.

In the foregoing water filter, the plurality of purification particles are purifying ceramic beads.

Thus, the water filter of the present invention may be coupled to a faucet by one end of the housing, allowing tap water to flow from the input portion toward the output portion. During the flowing process, the power generation unit is driven by the tap water via the spoiler unit, such that the power generation unit provides electric power needed by the sterilization unit, the sterilization unit sterilizes the tap water by means of photocatalysis, and the purification unit purifies the tap water and eliminates impurities in the tap water by means of neutralization, thereby achieving dual-effect water purification.

### BRIEF DESCRIPTION OF THE DRAWINGS

FIG. 1 is an appearance schematic diagram according to a preferred embodiment of the present invention.

FIG. 2 is an exploded schematic diagram according to a preferred embodiment of the present invention.

FIG. 3 is a schematic diagram of a state of use according to a preferred embodiment of the present invention.

### DETAILED DESCRIPTION OF THE INVENTION

To better understand the objects, features and effects of the present invention, detailed description of the present invention is given in specific embodiments with the accompanying drawings below.

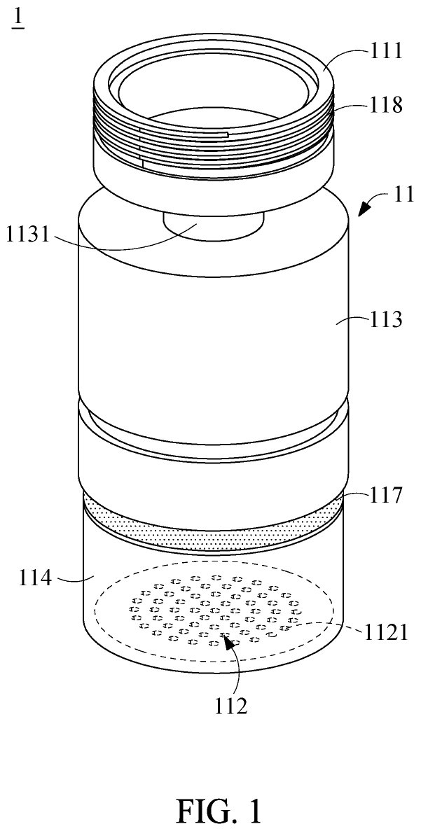

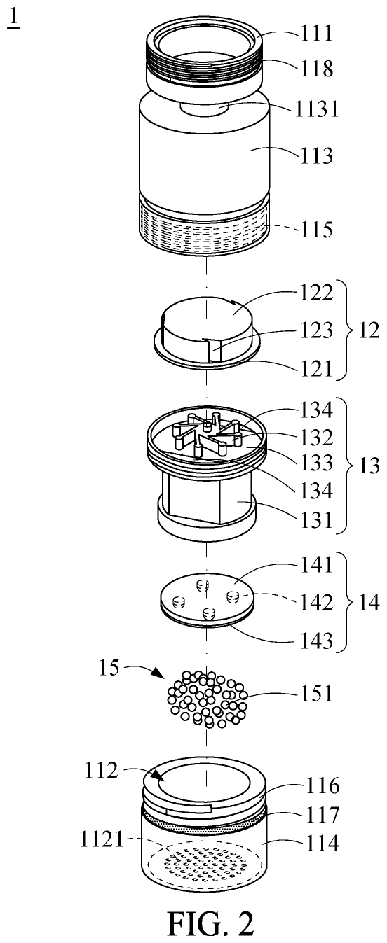

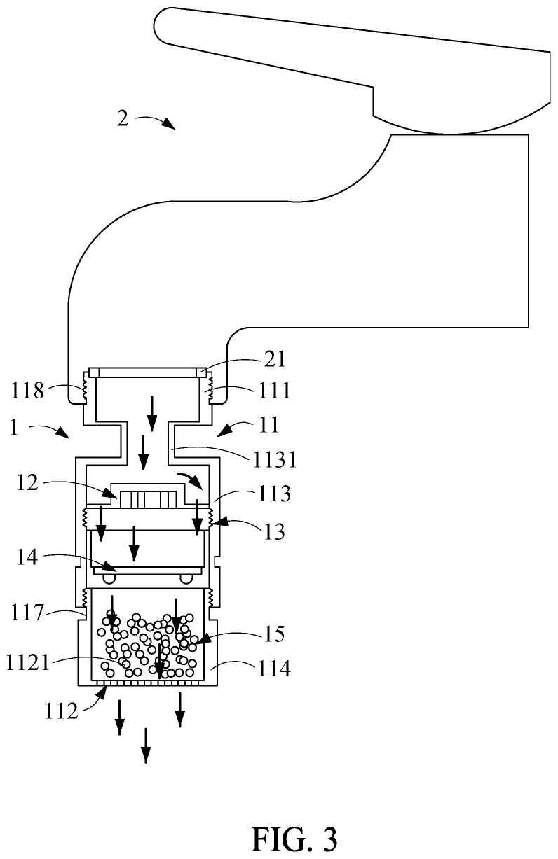

Referring to FIG. 1 to FIG. 3, as shown, the present invention provides a water filter. The water filter 1 includes a housing 11, a spoiler unit 12, a power generation unit 13, a sterilization unit 14 and a purification unit 15.

The housing 11 includes on two ends thereof an input portion 111 and an output portion 112 that are in communication with each other, respectively. The input portion 111 includes a first thread portion 118, enabling the input portion 111 to be detachably coupled to a faucet 2 by the first thread portion 118.

The spoiler unit 12 is provided in the housing 11, and corresponds to the input portion 111.

The power generation unit 13 is provided in the housing 11, and is coupled to the spoiler unit 12.

The sterilization unit 14 is provided in the housing 11 and is electrically connected to the power generation unit 13, and includes a sterilization light source.

The purification unit 15 is provided in the housing 11 and corresponds to the output portion 112 and the sterilization unit 13, and includes a plurality of purification particles 151.

To put to use, the water filter 1 may be coupled to a water outlet of the faucet 2 by the input portion 111 of the housing 11 coordinating with a waterproof washer 21. Thus, the input portion 111 may be coupled to the water outlet of the faucet 2 with the coordination of the first thread portion 118, enabling the input portion 111 to achieve an effect of easy removal and installation.

When the faucet 2 is turned on, the tap water enters the housing 11 from the input portion 111, passes through the spoiler unit 12, the power generation unit 13, the sterilization unit 14 and the purification unit 15, and flows toward the direction of the output portion 112. During the flowing process of the tap water, the tap water drives the power generation unit 13 via the spoiler unit 12, such that the power generation unit 13 provides electric power required by the sterilization unit 14 and activates the sterilization unit 14. The sterilization unit 14 emits a sterilization light source and sterilizes the tap water by means of photocatalysis, and the purification unit 15 purifies the tap water and eliminates impurities in the tap water by means of neutralization by the plurality of purification particles 151, achieving dual-effect water purification. The processed tap water is then output from the output portion 112 for use of a user.

In one embodiment of the present invention, the housing 11 includes an upper housing 113 and a lower housing 114. The upper housing 113 includes a second thread portion 115, and the lower housing 114 includes a third thread portion 116, such that the upper housing 113 may be detachably assembled by the second thread portion 115 with the third thread portion 116 of the lower housing 114, the input portion 111 is provided at the upper housing 113 and the output portion 112 is provided at the lower housing 114. Thus, the upper housing 113 and the lower housing 114 may achieve an effect of easy removal and installation with the coordination of the second thread portion 115 and the third thread portion 116 when the spoiler unit 12, the power generation unit 13, the sterilization unit 14 and the purification unit 15 are removed or assembled.

In one embodiment of the present invention, the lower housing 114 has a light transmissive area 117, which may correspond to the sterilization light source emitted by the sterilization unit 14. When the sterilization unit 14 is activated, the sterilization light source emitted by the sterilization unit 14 is displayed through the light transmissive area 117, allowing a user to learn from an outer side of the housing 11 whether the sterilization unit 114 is in an activated state.

In one embodiment of the present invention, the housing 1 further includes a neck portion 1131. The neck portion 1131 is in communication with the input portion 111 and the spoiler unit 12, and may be provided at the upper housing 113 on a position near the input portion 111. Thus, when tap water enters the housing 11 from the input portion 111, since the tap water flows from the input portion 111 having a large area to the neck portion 1131 having a smaller area, the flow speed of the tap water flowing to the spoiler unit 12 is increased by the neck portion 1131, so that the rotational speed of the power generation unit 13 driven by the tap water is increased, further increasing the amount of power generation of the power generation unit 13 to provide electric power required by the sterilization unit 14 and to activate the sterilization unit 14. Thus, the sterilization 14 is allowed to emit the sterilization light source to sterilize the tap water by means of photocatalysis, and the purification unit 15 then purifies the tap water and eliminates impurities in the tap water by means of neutralization by the plurality of purification particles 151, achieving dual-effect water purification. The processed tap water is then output from the output portion 112 for use of a user.

In one embodiment of the present invention, the output portion 112 consists of a plurality of through holes 1121, which are smaller than the plurality of purification particles 151. Thus, the plurality of purification particles 151 are prevented from falling out from the plurality of through holes 1121. Further, the time by which the tap water stays in the housing 11 is increased by the plurality of through holes 1121, allowing the sterilization unit 14 to effectively sterilize the tap water by means of photocatalysis, and the plurality of purification particles 151 of the purification unit 15 to purify the tap water and eliminate impurities in the tap water, achieving the object of reliable water purification.

In one embodiment, the power generation unit 13 includes an electromagnetic power generator 131 and a vane group 132. The vane group 132 is connected to the electromagnetic power generator 131. The electromagnetic power generator 131 has a limiting slot 133 on a top end thereof, and includes a channel portion 134 on each of both sides thereof, in a way that the spoiler unit 12 is provided at the limiting slot 133. Thus, the tap water is enabled to impel the van group 132 via the spoiler unit 12, and the vane group 132 then rotates to drive the electromagnetic power generator 131, such that the power generation unit 13 provides electric power required by the sterilization unit 14 and activates the sterilization unit 14, and the tap water is allowed to flow from the channel portions 134 to the sterilization unit 14. The sterilization unit 14 then emits the sterilization light source to sterilize the tap water by means of photocatalysis, and the purification unit 15 then purifies the tap water and eliminates impurities in the tap water by means of neutralization by the plurality of purification particles 151, achieving dual-effect water purification. The processed tap water is then output from the output portion 112 for use of a user.

In one embodiment of the present invention, the spoiler unit 12 includes an assembly portion 121, a hood 122 and a plurality of spoiler entrances 123. The assembly portion 121 encircles the bottom of the hood 122, the plurality of spoiler entrances 123 are provided on the periphery of the hood 122, and the assembly portion 121 is provided in the limiting slot 133 of the electromagnetic power generator 131. Further, the hood 122 covers the vane group 132, the plurality of spoiler entrances 123 are in communication with the plurality of channel portions 134, and the plurality of spoiler entrances 123 are disposed along a tangent direction of the vane group 132. Accordingly, the tap water with a flow speed increased by the neck portion 1131 enters the hood 122 from the plurality of spoiler entrances 123 and impels the vane group 132, and the speed of the tap water impelling the vane group 132 is further increased since the plurality of spoiler entrances 123 are disposed along the tangent direction of the vane group 132. Thus, the rotational speed of the tap water impelling the vane group 132 is increased, and the vane group 132 rotates and drives the electromagnetic power generator 131, such that the power generation unit 13 provides electric power required by the sterilization unit 14 and activates the sterilization unit 14, and the tap water is allowed to flow from the channel portions 134 to the sterilization unit 14. The sterilization unit 14 then emits the sterilization light source to sterilize the tap water, and the purification unit 15 then purifies the tap water and eliminates impurities in the tap water by the plurality of purification particles 151, achieving dual-effect water purification. The processed tap water is then output from the output portion 112 for use of a user.

In one embodiment of the present invention, the sterilization unit 14 includes a control module 141, at least one sterilization lamp 142 and a waterproof glue layer 143. The control module 141 is provided at the bottom of the electromagnetic power generator 131 provided at the power generation unit 13, and is capable of controlling an operating mode of the sterilization lamps 142. The sterilization lamps 142 are provided in an electrically connected manner on one surface of the control module 141, so as to emit the sterilization light source. The waterproof glue layer 142 is provided at a portion of a surface of the control module 141 and covers the sterilization lamps 142, so as to prevent the control module 141 and the sterilization lamps 142 from damage by moisture. The control module 141 may be a circuit board, a logical circuit, a single chip or a combination thereof. The sterilization lamps 142 may be UVC light sources. Thus, when the power generation unit 13 provides electrical power required by the sterilization unit 14, the control module 141 may be activated to control the operating mode of the sterilization lamps 142, enabling the sterilization lamps 142 to sterilize the tap water by the sterilization photocatalysis light source emitted.

In one embodiment of the present invention, the plurality of purification particles 151 may be purifying ceramic beads (for example, silver ion ceramic beads). Thus, the tap water is purified and impurities thereof are eliminated by means of neutralization by the plurality of purification particles 151, and the sterilization unit 14 also effectively sterilizes the tap water, achieving the object of reliable water purification.

While the invention has been described by way of example and in terms of the embodiments, it is to be understood that the embodiments are for illustrating the present invention and are not to be construed as limitations to the scope of the present invention. It should be noted that, equivalent changes and modifications are to be encompassed within the scope of the present invention. Therefore, the protection scope of the present invention is to be accorded with the broadest interpretation of the appended claims.

What is claimed is:

1. A water filter, comprising:
a housing, comprising on two ends thereof an input portion and an output portion that are in communication with each other, respectively, the input portion comprising a first thread portion, enabling the input portion to be detachably coupled to a faucet by the first thread portion;
a spoiler unit, provided in the housing and corresponding to the input portion;
a power generation unit, provided in the housing and coupled to the spoiler unit;
a sterilization unit, provided in the housing and electrically connected to the power generation unit, the sterilization unit comprising a sterilization light source; and
a purification unit, provided in the housing and corresponding to the output portion and the sterilization unit, the purification unit comprising a plurality of purification particles.

2. The water filter of claim 1, wherein the housing has a light transmissive area corresponding to the sterilization light source.

3. The water filter of claim 1, wherein the housing further comprises a neck portion in communication with the input portion and the spoiler unit.

4. The water filter of claim 1, wherein the power generation unit comprises an electromagnetic power generator and a vane group, the vane group is connected to the electromagnetic power generator, the electromagnetic power generator has a limiting slot on a top end thereof and a channel portion on each of both sides thereof, the spoiler unit comprises an assembly portion, a hood and a plurality of spoiler entrances, the assembly portion encircles a bottom of the hood, the plurality of spoiler entrances are provided on a periphery of the hood, the assembly portion is provided in the limiting slot of the electromagnetic power generator, the hood covers the vane group, the plurality of spoiler entrances are in communication with the channel portions, and the plurality of spoiler entrances are disposed along a tangent direction of the vane group.

5. The water filter of claim 1, wherein the sterilization unit comprises a control module, at least one sterilization lamp and a waterproof glue layer, the control module is provided at a bottom of the power generation unit, the sterilization lamps are provided in an electrically connected manner on one surface of the control module such that the sterilization lamps are capable of emitting a sterilization light source, and the waterproof glue layer is provided on one surface of the control module to expose the sterilization lamps.
